(19) United States
(12) Patent Application Publication
(10) Pub. No.: US 2018/0008149 A1
(43) Pub. Date: Jan. 11, 2018

(54) Systems and Methods of Body Temperature Measurement

(71) Applicant: General Electric Company, Schenectady, NY (US)

(72) Inventor: Otto Valtteri Pekander, Helsinki (FI)

(73) Assignee: General Electric Company, Schenectady, NY (US)

(21) Appl. No.: 15/203,375

(22) Filed: Jul. 6, 2016

(51) Int. Cl. A61B 5/01; A61B 5/00; G01K 13/00

(52) U.S. Cl. CPC A61B 5/01; G01K 13/002; A61B 5/0008; A61B 5/6832; A61B 5/7282

(57) ABSTRACT

Systems and methods of body temperature measurement obtain a first temperature from a first temperature sensor. The first temperature sensor is separated from a second temperature sensor by a first insulator. A second temperature is obtained from the second temperature sensor. A heat transfer rate between the first temperature and the second temperature sensor is calculated and a body temperature determined from the heat transfer rate.

### BACKGROUND

The present disclosure relates to the field of temperature measurement. More specifically, the present disclosure relates to sensor systems and methods of using sensor systems to measure body temperature.

A patient's body temperature can give a physician important information about the physiological state of the patient. Typically, when body temperature is measured, the body temperature is an external body temperature which is used to approximate or represent the patient's internal or core body temperature. Absent an external heating source, a patient's body heat is generated internally and conducted outward to the skin and extremities. However, external temperature measurements are limited in their approximation of core body temperature as muscle, fat, and skin tissue act as insulators to an unknown degree specific to each patient and location on the patient. Thus, the external temperature of generated body heat is less than the patent's core body temperature, but by an unknown amount.

Known attempts to better represent core body temperature include placement of the thermometer or other temperature sensor at a location “internal” from the exterior of the patients skin, for example, in the ear, under the tongue, in a closed armpit, or in the rectum. However, the same problems as noted above exist with these locations as the difference between the measured external temperature is still less than the patient's internal body temperature by an known amount, although this amount may be lessened by the use of those techniques. Furthermore, each of those locations are more intrusive to the patient and are inconvenient for a sensor location for repeated measurement of body temperature. A sensor location is more convenient on widely exposed areas of the patient's skin, for example the forehead, chest, back, arms, or legs. However, it is these exposed locations which are most susceptible to unpredictability between the difference in core body temperature and the measured skin temperature.

One available solution to non-invasively measure core temperature attempts to create a perfect insulator between the skin and air in the sensor. This approach involves continuously heating the sensor surface until an equilibrium is reached and there is no heat transfer. The temperature at which this equilibrium is reached is identified as the patient's core body temperature. However, this approach requires continuously applying heat to the patient's skin as well as creates high energy demands to operate such a heating source. Both of these results may be undesirable for use in a wireless non-invasive temperature sensor.

Therefore, improved solutions for the determination of the core body temperature are desired in the field of patient monitoring.

### BRIEF DISCLOSURE

An exemplary embodiment of a method of measuring body temperature includes obtaining a first temperature from a first temperature sensor. A second temperature is obtained from a second temperature sensor, the first and second temperature sensors being separated by a first insulator which has a first conductivity value. A third temperature is obtained from a third temperature sensor and a fourth temperature is obtained from a fourth temperature sensor. The third and fourth temperature sensors are separated by a second insulator which has a second conductivity value. The second conductivity value is different from the first conductivity value. A first heat transfer rate between the first temperature sensor and the second temperature sensor is calculated. A first heat transfer rate between the third temperature sensor and the fourth temperature sensor is calculated. A body temperature is determined from the first heat transfer rate and the second heat transfer rate.

An exemplary embodiment of a system for measuring body temperature includes a first sensing module and a second sensing module. The first sensing module includes a first temperature sensor and a second temperature sensor. A first insulator is disposed between the first temperature sensor and the second temperature sensor. The first insulator has first conductivity value. The second sensing module includes a third temperature sensor and a fourth temperature sensor. A second insulator is disposed between the third temperature sensor and the fourth temperature sensor. The second insulator has a second conductivity value.

### BRIEF DESCRIPTION OF THE DRAWINGS

FIG. 1 is a schematic diagram of an exemplary embodiment of a body temperature sensor.

FIG. 2 is a system diagram of a sensing system.

FIG. 3 is a schematic diagram of an addition exemplary embodiment of a body temperature sensor.

FIG. 4 is a flow chart that depicts an exemplary embodiment of a method of measuring body temperature.

FIG. 5 is a flow chart that depicts a more detailed exemplary embodiment of a method of measuring body temperature.

### DETAILED DISCLOSURE

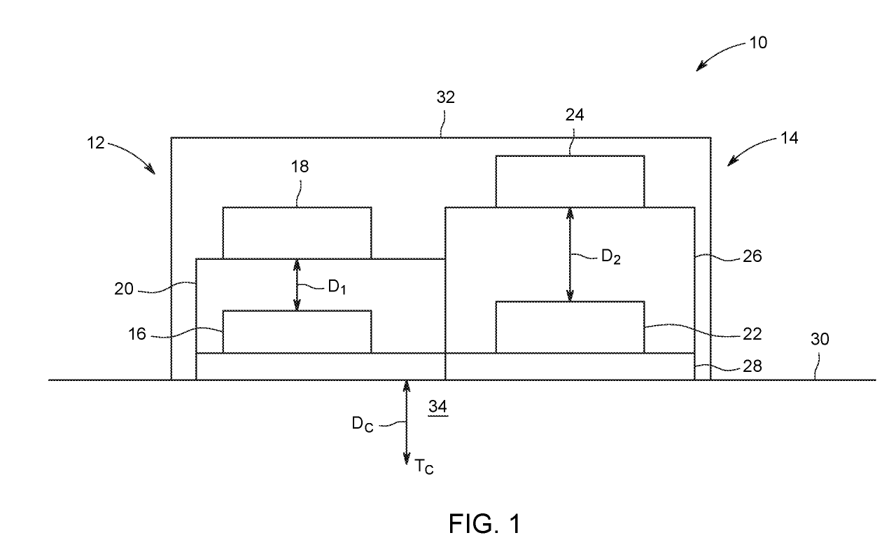

FIG. 1 is a schematic diagram of an exemplary embodiment of a temperature sensor 10. It will be recognized that FIG. 1 is schematic in nature and is intended for the purpose of disclosing and describing features of an embodiment of the temperature sensors rather than specific sizes or measurements of such features. The temperature sensor 10 and other embodiments as described herein are suited for the exemplary use of non-invasively determining a body temperature, for example a core body temperature.

The temperature sensor 10 exemplarily includes a sensing module 12 and a sensing module 14. It will be recognized that FIG. 1 depicts an exemplary embodiment wherein the temperature sensor 10 comprises both of the sensing modules 12, 14 in a single unit. In other embodiments, it will be recognized that each of the sensing modules 12, 14 may be separate components although communicatively connected as described herein.

Sensing module 12 includes a sensor 16 and a sensor 18 separated by an insulator 20. The insulator 20 has a conductivity represented by a conductivity value and is of a thickness d₁ between the sensor 16 and the sensor 18 as will be described in further detail herein.

Sensing module 14 includes sensor 22 and sensor 24 which are separated by an insulator 26. The insulator 26 has a conductivity represented by a conductivity value and has a thickness d₂ between the sensor 22 and the sensor 24. In a non-limiting embodiment, each of the sensors 16, 18, 22, 24 are I2C digital temperature sensors. In a further non-limiting embodiment, the temperature sensors have an accuracy of 0.05° C. It will be recognized that these temperature sensors are exemplary and not intended to be limiting on the scope of temperature sensors which may be used in embodiments as disclosed herein. Non-limiting examples of temperature sensors include thermocouples, thermistors, and radiation detectors.

Embodiments of the temperature sensor 10 further include an adhesive layer 28 which is exemplarily applied across the sensor 16 and sensor 22 and facilitates to secure the temperature sensor 10 to the skin 30 of the patient. A protective coating 32 is exemplarily applied across an opposite side of the temperature sensor 10, for example across the sensor 18 and sensor 24. The protective coating 32 may exemplarily be a polymeric film, and as explained in further detail herein, a material suitable for protecting the sensor 18 and sensor 24 from air circulation. In embodiments of the temperature sensor, the sensing module 12 and sensing module 14 may be either physically separated or thermally separated by an insulator (not depicted) of a low conductivity. This can help to maintain the differences in the thermal systems of sensing module 12 and sensing module 14 by educing thermal cross-contamination.

As mentioned above, additional embodiments of the sensor 10 may be implemented with separate units comprising sensing module 12 and sensing module 14. In such embodiments, for example, it will be recognized that separate adhesive protective coatings over the sensor would be used.

As exemplarily depicted in FIG. 1, a generally unknown amount of subcutaneous tissue 34 exists between the surface of the skin 30 and the patient body core, for example where the temperature is the patient core temperature (T_{C}) This layer of subcutaneous tissue may include skin, fat, muscle, bone, blood and blood vessels, and/or other organs. Because a suitable sensor must be robust and able to be used with patients of varying body types, sizes, and physical make up, the sensor must operate independent from the content and the thickness (d_{C}) of this subcutaneous tissue. In most instances, the actual thickness d_{C} and the exact make up and resulting thermal conductivity (k) of the subcutaneous tissue will be known, such that the conductivity value is similarly unknown. The subcutaneous tissue 34 acts as an insulator between the body core temperature (T_{C}) and the external temperature at the surface of the skin 30. Embodiments of the sensor and methods of measuring body temperature as disclosed herein overcome these unknowns and challenges to determining a more representative value of body temperature with a non-invasive sensor.

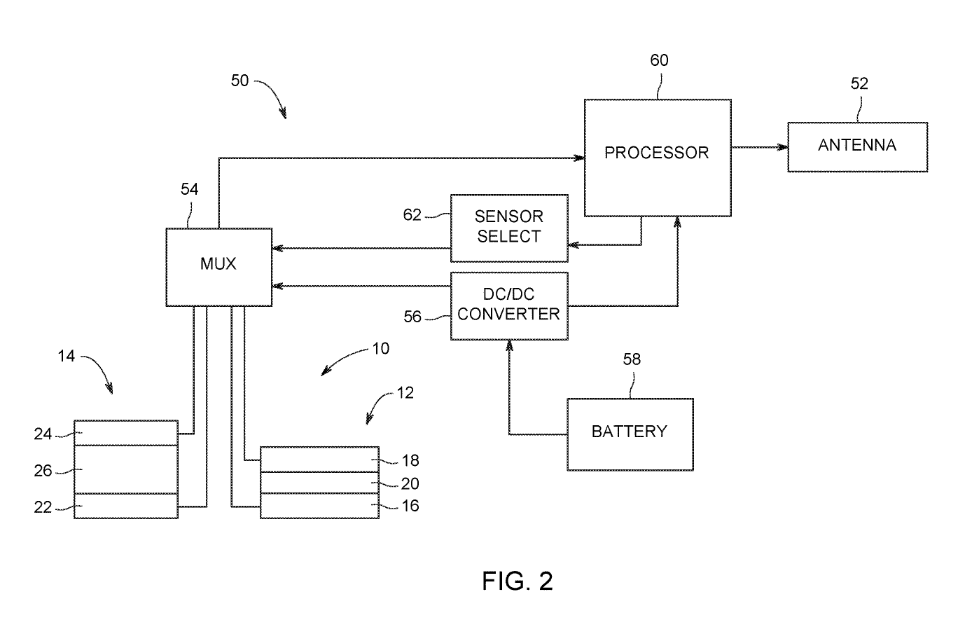

FIG. 2 depicts a further exemplary embodiment of a sensing system 50 which includes an exemplary embodiment of the temperature sensor 10. The temperature sensor 10 exemplarily includes the sensing module 12 and the sensing module 14 as described above with respect to FIG. 1. As noted above, the sensing modules 12, 14 along with the entirety of the sensing system 50 may be part of a single sensor device secured to a patient. It will be recognized that in additional exemplary embodiments, the sensing modules 12, 14 may be separate components which may each include their own adhesive layers (not depicted) and protective coatings (not depicted) and be positioned at different anatomical locations on the patient. In an exemplarily embodiment, the sensing module 12 and sensing module 14 may be positioned at equivalent anatomical positions on the patient, for example, both temples of the head, both earlobes, both shoulder blades, both thighs, etc. In this respect, the sensing modules 12, 14 are positioned at locations that are anatomically equal from one another to the internal point in the patient's body from which the core temperature (T_{C}) is presumed.

In exemplarily embodiments, the sensing system 50, in use, is secured to the patient, for example, integral part to the temperature sensor 10. Particularly in an embodiment wherein both sensing module 12 and sensing module 14 are combined in the same unitary device, the rest of the sensing system 50 may be similarly integrated. In an exemplary embodiment, the sensing system 50 wirelessly transmits the determined body temperature via an antenna 52, for example, on a medical body area network (MBAN) to a MBAN monitoring device or hub (not depicted) as known in the field to receive wirelessly transmitted data acquired from a patient using a wireless sensor.

In additional embodiments, the sensing modules 12, 14 may be separate components. In one example, the rest of the sensing system 50 may be integrated with one or the other of the sensing modules 12, 14. In a further example, the rest of the sensing system 50 may be a separate device positioned local to the patient. In such an event, the sensing modules 12, 14 may be communicatively connected to the rest of the sensing system 50 via wired or wireless connections therebetween. If such communicative connections are wireless, the sensing modules 12, 14 may further include a power source and antenna (not depicted) to facilitate such wireless transmission.

The sensing system 50 exemplarily includes a multiplexer 54 which is communicatively connected, either wired or wirelessly as described above, to the sensors 16 and 18 of the sensing module 12 and is similarly communicatively connected to the sensors 22 and 24 of the sensing module 14. The multiplexer 54 is exemplarily connected to a D_{C}/D_{C} converter 56 which is connected to a power supply 58 which is exemplarily a battery. The D_{C}/D_{C} converter 56 provides an input voltage (e.g. 1.8 volts) to the sensors 16, 18, 22, 24 through the multiplexer 54. The multiplexer 54, in return, receives back the returned output voltages from each of the sensors.

A processor 60 is communicatively connected to the multiplexer 54 as well as to a sensor selector 62. The sensor selector 62 provides a sensor selection signal to the multiplexer 54 to select through the signals of each of the sensors which the multiplexer 54 provides to the processor 60. It will be recognized that in an alternative embodiment, all of the temperature signals may be simultaneously received by the processor without a multiplexer if the processor is capable of receiving a suitable number of channels of information. In one of these ways, the processor 60 exemplarily receives temperature signals from each of the sensors. The processor 60 is operable to obtain a temperature measurement from each of the received sensor signals. These temperatures are exemplarily indicated as T₁ from sensor 16, T₂ from sensor 18, T₃ from sensor 22, and T₄ from sensor 24. However, it will be recognized that this association and numbering of the temperature signals and associated sensors is not intended to be limiting and that other associations between the sensors and particular temperature measurement values may be made within the scope of the present disclosure. In an exemplary embodiment, each of the temperature measurement may be expected to be different values from each other due to the design of the sensing modules 12, 14 themselves.

The processor 60 operates in the manners as disclosed in further detail herein, for example, by executing computer readable code stored in computer readable storage located at the processor 60 or communicatively connected to the processor 60. Computer readable code may be divided into one or more sub routines which may be stored together or apart in one or more executable files as will be recognized by a person of ordinary skill in the art.

Thus, the processor 60, upon execution of the computer readable code operates with the equations and functionalities as described in further detail herein to determine a body temperature from the received sensors signal as well as additional information as described herein which may be similarly stored in a computer readable medium communicatively connected to the processor 60.

As will be described in further detail herein, in an exemplary embodiment, the core temperature is assumed to be identical at a common point between both measurement locations (e.g. the positions at which the sensing module 12 and sensing module 14 are secured to the patient) which may be similarly represented by the positions of sensor 16 and sensor 22, respectively. Additionally, thermal insulation caused by the subcutaneous tissue, assumed to have identical thickness and properties between both measuring locations and the location of the core temperature is also unknown. As noted above, the sensing modules includes insulator 20 of a known thickness d, and thermal conductivity k₁ and insulator 26 which has a known thickness d₂ and thermal conductivity k₂. The conductivity value of the insulator may be expressed in terms of the thermal conductivity and the thickness of the insulator (k/d). In exemplary embodiments, the thermal conductivity (k) of the materials of insulator 20 and insulator 26 may be the same, although the resulting overall conductivity value of the insulators may be different due to differences in the thicknesses of the two insulators. It will be recognized that a material at any thickness will have a higher conductance than the conductance through that same material at twice the thickness. In an alternative embodiment, two different insulators of known thermal conductivities may be used. In such examples the thicknesses of insulators 20 and 26 may be the same and/or different. It will be recognized that while the insulators are described herein with respect to conductivity, that conductance and resistance are reciprocally related and therefore may be equivalently used. It will further be recognized by a person of ordinary skill in the art that the thermal conductance of an insulator may be expressed as a “u value” while a thermal resistance of an insulator may be represented as a “r value”. In an exemplary embodiment, as described in further detail herein, a heat transfer rate through the sensing module 12, for example, between sensor 16 and sensor 18 may be expressed with the following equation:

$${W}_{1} = \frac{{k}_{1}}{{d}_{1}} ( {T}_{2} - {T}_{1} ) \qquad ( 1 a )$$

wherein k₁ is the thermal conductivity of the insulator 20 d₁ is the thickness of insulator 20, T₁ is the measurement of the temperature at sensor 16 and T₂ is the measurement of the temperature at sensor 18.

If an assumption is made that the temperature at the body core (T_{C}) is to be treated as a steady state system for the purposes of measurement, then the heat transfer rate within the body from the body core to sensor 16 is the same heat transfer rate as between sensor 16 and sensor 18. Thus, the heat transfer rate from the body core to sensor 16 can be represented as:

$${W}_{1} = \frac{{k}_{t}}{{d}_{t}} ( {T}_{1} - {T}_{C} ) \qquad ( 1 b )$$

Wherein kₜ is the thermal conductivity of the body tissue and dₜ is the thickness of the body tissue to the location of the core temperature.

In sensing module 14, the heat transfer rate (W₂) can be modeled as:

$${W}_{2} = \frac{{k}_{2}}{{d}_{2}} ( {T}_{4} - {T}_{3} ) \qquad ( 2 a )$$

wherein k₂ is the thermal conductivity of insulator 26 d₂ is the thickness of insulator 26, T₃ is the temperature measured at sensor 22, and T₄ is the temperature measured at sensor 24.

For the reasons as noted above the heat transfer rate W₂ is the same between the body core and the sensor 22 as it is from the sensor 22 to sensor 24. Thus, the heat transfer rate W₂ can also be represented as:

$${W}_{2} = \frac{{k}_{t}}{{d}_{t}} ( {T}_{3} - {T}_{C} ) \qquad ( 2 b )$$

While it is assumed that the conductivity of that subcutaneous tissue between the body core and the sensing modules is the same, because the conductance of insulator 20 is different from the conductance of insulator 26, that the heat transfer rates within the two systems are different. In this respect, a system that includes a sensing module with an insulator with a higher conductance will have a higher heat transfer rate while a system that includes assembly module with an insulator with a lower conductance will have a lower heat transfer rate. These differences in heat transfer rates between the two systems result in differences in the temperatures measured at each of the temperature sensors. One exemplary experimental measurement found that a difference between the conductance of the insulators of two sensing nodules produced a 0.2° C. difference between the temperatures measured by otherwise similarly positioned sensors (e.g. sensor 16 and sensor 22).

The above equations 1a-2b can be solved for the unknown conductance of the subcutaneous tissue 34 between the body core and the sensing modules and represented in equation 3:

$$\frac{{k}_{t}}{{d}_{x}} = \frac{{k}_{1}}{{d}_{1}} \frac{( {T}_{2} - {T}_{1} )}{( {T}_{1} - {T}_{C} )} = \frac{{k}_{2}}{{d}_{2}} \frac{( {T}_{4} - {T}_{3} )}{( {T}_{3} - {T}_{C} )} \qquad ( 3 )$$

Finally, the relationships of equation 3 can be solved for the core temperature (T_{C}) as represented in terms of heat transfer rates and temperatures in equation 4a or in terms of only the measured temperatures and insulation in equation 4b:

$$\begin{matrix}{T}_{C} = \frac{\frac{{W}_{1}}{{W}_{2}} {T}_{3} - {T}_{1}}{\frac{{W}_{1}}{{W}_{2}} - 1} & ( 4 a ) \\ {T}_{C} = \frac{{T}_{1} + {T}_{3} ( \frac{\frac{{k}_{1}}{{d}_{1}}}{\frac{{k}_{2}}{{d}_{2}}} ) ( \frac{{T}_{1} - {T}_{2}}{{T}_{4} - {T}_{3}} )}{1 + ( \frac{\frac{{k}_{1}}{{d}_{1}}}{\frac{{k}_{2}}{{d}_{2}}} ) ( \frac{{T}_{1} - {T}_{2}}{{T}_{4} - {T}_{3}} )} & ( 4 b )\end{matrix}$$

Thus, given the assumptions made as identified above, the core body temperature can be determined from the measurements of the sensing system as described above.

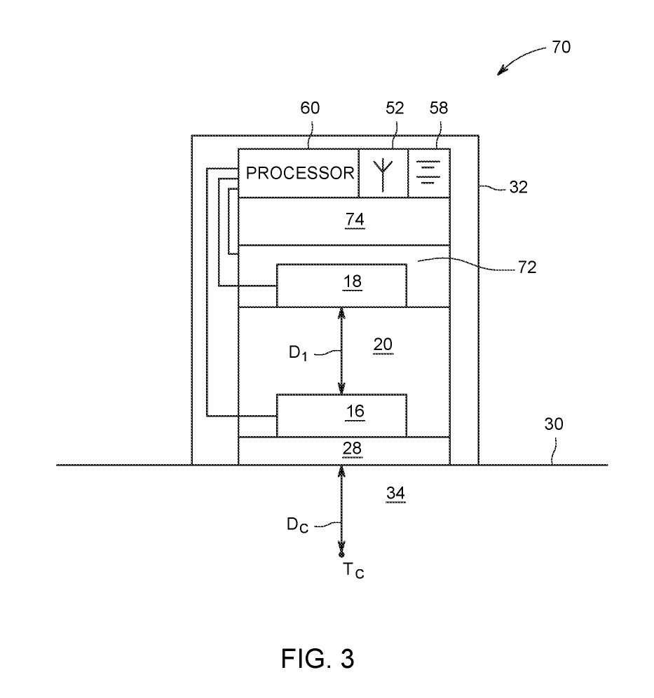

FIG. 3 depicts a still further exemplary embodiment of a sensor system. 70. As noted above, in exemplary and nonlimiting embodiments, the sensor system 70 may be embodied in a single wireless sensor configured to be secured to the skin of the patient. Therefore, FIG. 3 exemplarily depicts a sensor system 70 in which the processor 60, antenna 52, and battery 58 are exemplarily all contained within the sensor package, or example covered by protective coating 32.

While like reference numerals have been used in FIG. 3 to reference structures similar to those previously described, the sensor system 70 of FIG. 3 further depicts an exemplary embodiment in which a single sensing module 12 in which an insulator 20 separates sensor 16, 18 and as described in further detail herein at least two temperature measurements are obtained by the processor 60 from each of the sensors 16, 18. The processor 60 is exemplarily communicatively connected to the antenna 52 to broadcast the body temperatures as determined by the processor 60 to a monitoring device or data hub associated with a patient monitoring system of which the sensor system 70 is a part.

In addition, the sensor system 70 includes a heating device 74, which will exemplarily be a resistive film or other resistive heating element, it will be recognized that other types of heating devices may be used within scope of the present disclosure. An insulator 76 is positioned over the heating device 74 and sensor 18, exemplarily disposed between the heating device 74 and the processor 60, battery 58, and antenna 52. The heating device 74 is operatively connected to the processor 60 as well as to the battery 58 and is used in a calibration phase or calibration routine as operated by the processor 60 in acquiring the first and second temperatures from the sensor 16 and sensor 18 as disclosed herein. The calibration routine may be conducted exemplarily upon placement of the sensor 70 on the patient, upon making a first measurement of body temperature with the sensor or at any other time during use, particularly a monitoring system or clinician determines that it may be necessary to recalibrate the sensor, for example if the sensor is moves or repositioned or it is believed that the patient's core body temperature may have significantly changed since a previous calibration.

As will be described in further detail herein, the heating device 72 is operated by the processor 60 to generate heat. The heat generated from the heating device 72 is transferred to the sensor 18 and through the insulator 20 to the sensor 16. In an embodiment wherein the heating device 72 is in thermal contact with the sensor 18, a measurement of temperature at the sensor 18 will reflect the temperature of the in heating device 72. While the temperature at the sensor 16 will reflect the combined effect of the heating device 72 and the heat produced by the patient's body. The heating device 72 operates and a temperature at the sensor 18 is monitored. In one embodiment the temperatures ant the sensors are measured at any point once the heating device 72 has heated the system above ambient. In another embodiment, the temperatures are measured when the sensor 18 reads a predetermined value. In a still further exemplary embedment the temperature at sensor 18 is monitored until it reaches a steady state or equilibrium at which point the temperatures at the sensors are measured.

In any event, at some point while the heating device 72 is operating to heat the sensing system, first and second temperatures are measured from the sensor 16 and 18, respectively. These are the calibration measurements and are used in conjunction with further third and fourth temperature measurements obtained at a time when the patient's body core temperature is to be determined.

The processor 60 may operate the heating device 72 to stop producing heat. Particularly, in a wireless sensing device, operation of the heating device 72 may use energy from the battery 58 and therefore, in an embodiment, use of the heating device 72 may be limited to the calibration routine to conserve battery power.

Once the heating device 72 is turned off, the temperatures at the sensors 16 and 18 will decrease to reflect the decrease of heat in the system. Eventually, the temperatures at the sensor 16 and 18 will reach an equilibrium which reflects the heat provided by the patient's body, and more specifically the heat provided from the patient's body core. The insulator 74 and the protective film 32 help to thermally isolate the sensors 16, 18 from the ambient temperature.

In an exemplary embodiment, the processor 60 monitors the temperatures at the sensors 16, 18 until the temperatures at these sensors reach an equilibrium along a steady state reflective of the heat provided by the body core. When this equilibrium is reached, third and fourth temperature measurements may be obtained respectively from temperature sensor 16 and temperature sensor 18 at any time that a measurement of the patient body temperature is desired.

In accordance with the method as disclosed herein, the heat transfer rate during the calibration phase may be represented as:

$$\frac{( {T}_{x} - {T}_{2} )}{( {R}_{x} - {R}_{1} )} = \frac{( {T}_{1} - {T}_{2} )}{{R}_{1}} \qquad ( 5 a )$$

wherein Rₓ is the thermal resistance of the body tissue and R₁ is the thermal resistance of the insulator 20. It will be recognized that as mentioned above, thermal resistance is the inverse of thermal conductance and therefore thermal resistance may exemplarily represent thermal resistivity across a particular thickness of tissue or insulator.

The heat transfer rate during the measurement phase is similarly represented as:

$$\frac{( {T}_{x} - {T}_{4} )}{( {R}_{x} - {R}_{1} )} = \frac{( {T}_{3} - {T}_{4} )}{{R}_{1}} \qquad ( 5 b )$$

Equations 5a and 5b may be solved for core temperature (T_{C} when T_{C} is represented as a relation based solely upon the measured temperatures and the differential temperatures between the electrodes 16, 18. This is exemplarily represented in equation 6:

$${T}_{C} = \frac{{T}_{1} - {T}_{4} \frac{( {T}_{1} - {T}_{2} )}{( {T}_{3} - {T}_{4} )}}{\frac{( {T}_{1} - {T}_{2} )}{( {T}_{3} - {T}_{4} )} - 1} \qquad ( 6 )$$

In the manner described above, an embodiment of the sensor system in accordance with the present disclosure may be used to determine a body temperature of the patient with measurements from two temperature sensors. After the sensor system has been calibrated, further determinations of body temperature may be made with only acquiring a new set of temperature measurements from temperature sensor 16 and temperature sensor 18.

In a still further embodiment in accordance with the other embodiments disclosed herein, heat transfer rate may be measured with a heat flux sensor. It will be recognized that heat transfer is expressed as power (Watts) while heat flux is expressed Watts per meter squared. In embodiments the body temperature may be calculated as disclosed herein from the measured heat transfer rates and/or may be calculated in combination with one or more of the temperature measurements as described in the present application.

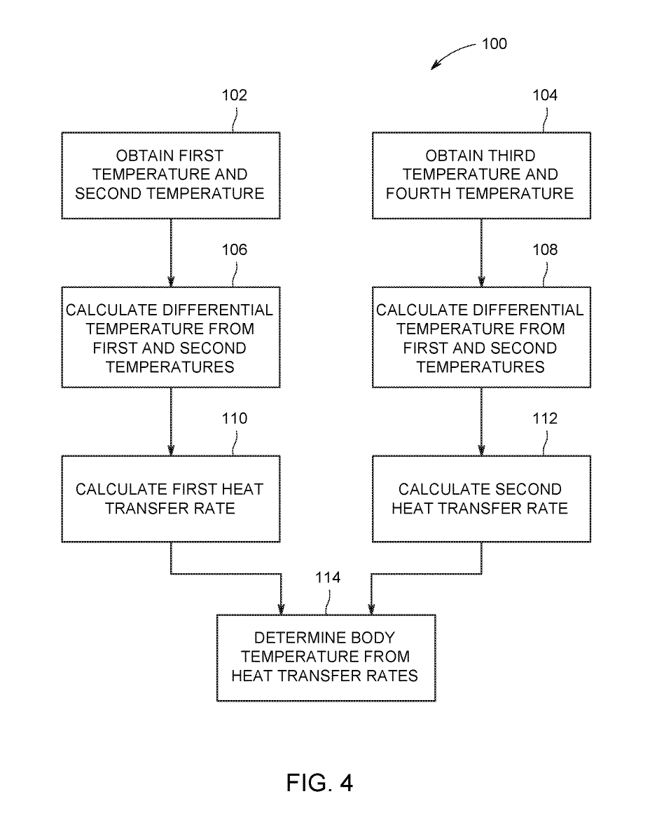

FIG. 4 is a flow chart that depicts an exemplary embodiment of a method 100 of measuring body temperature. The method 100 will be described in further detail herein with respect to an exemplary embodiment of the method. It will be recognized that the described embodiment is merely exemplary of embodiments within the scope of the disclosed method, other embodiments of the method 100, including, but not limited to the method 200 as disclosed in further detail herein may be further exemplary embodiments of the method 100.

The method 100 includes at 102 that a first temperature and a second temperature are obtained. The first temperature may be obtained from a sensor signal of a first temperature sensor and the second temperature may be obtained from a sensor signal from a second temperature sensor. The respective sensor signals may be received and processed by a processor to produce an output of the first and second temperatures. In an exemplary embodiment, the first and second temperature sensors are separated by a first insulator which has a first conductance valve. The first conductance value is exemplarily known and is based upon a thermal conductivity of the material of the first insulator at a thickness of the first insulator.

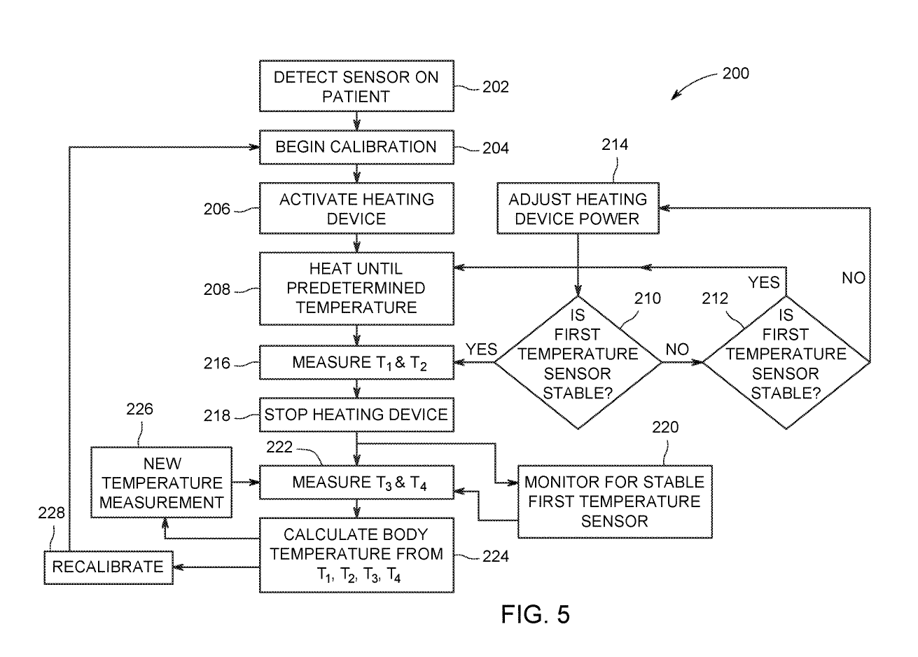

At 104 a third temperature and a fourth temperature are obtained. Similar to that of 102, the third temperature is exemplarily obtained from a sensor signal of a third temperature sensor and the fourth temperature is obtained from a sensor signal of a fourth temperature sensor. However, it will be recognized that in other embodiments, the third temperature and the fourth temperature may be obtained from the same temperature sensors from which the first and second temperatures were obtained, for example as in the exemplary embodiment of the method 200 described with respect to FIG. 5 herein. The respective sensor signals may be received and processed by the processor to produce an output of the third and fourth temperatures. The third and fourth temperature sensors may exemplarily be separated by a second insulator which has a second conductance value. The second conductance value is exemplarily known and is based upon a thermal conductivity of the material of the second insulator and a thickness of the second insulator.

In exemplary embodiments, the first insulator and the second insulators have different conductance values. The difference in the conductance values may be due to the first insulator and the second insulator having different thicknesses of the same material or the first insulator and the second insulator may be constructed of different materials and exemplarily have the same thickness. However, it will be recognized that other combinations of insulator material and thickness may be used in accordance with the present disclosure to arrive at a first insulator of a known first conductance value and a second insulator of a known second conductance value in which the first conductance value and the second conductance value are different.

Next, at 106 a differential temperature between the first temperature and the second temperature is calculated. This may exemplarily be a differential temperature across the first sensing module, which e.g. may be the differential temperature across the first insulator between the first sensor and the second sensor. This differential temperature may be calculated as the differential temperature between the obtained first temperature and the obtained second temperature from 102. The differential temperature across the first insulator is representative of the amount of heat that is conducted through the first insulator between the location of the first temperature sensor to the location of the second temperature sensor.

At 108 a differential temperature between the third temperature and the fourth temperature is calculated. This may exemplarily be a differential temperature across the second sensing module, which e.g. may be the differential temperature across the second insulator between the third sensor and the fourth sensor. This differential temperature may be calculated as the differential temperature between the third temperature and the fourth temperature. Thus, the differential temperature across the second insulator is the amount of heat delivered across the second insulator between the third temperature sensor to the fourth temperature sensor.

At 110 a first heat transfer rate is calculated. The first heat transfer rate is exemplarily the heat transfer rate across the first sensing module/first insulator is calculated. Exemplarily, this may be calculated using Equation 1a which represents the heat transfer rate based upon a known conductance of the first insulator and the differential temperature between the first and second sensors on either side of the first insulator. It will be recognized that the more conductive that the first insulator is, the greater the heat transfer rate will be across the first insulator.

Similarly at 112, a second heat transfer rate is calculated. The second heat transfer rate is exemplarily the heat transfer rate across the second sensing module/second insulator is calculated. Exemplarily, the heat transfer rate across the second insulator may be calculated using Equation 2a as described above, which represents the heat transfer rate across the second insulator based upon a known conductance of the second insulator and the differential temperature, between the third and fourth sensors on either side of the second insulator.

Next, at 114 the body temperature is determined based upon the heat transfer rate across the first insulator calculated at 110 and the heat transfer rate across the second insulator calculated at 112. This may exemplarily be determined using Equation 4 as described above. In exemplary embodiments, the body temperature is further determined based upon the calculated heat transfer rates in addition to the first temperature obtained at 102 and the third temperature obtained at 104. In exemplary embodiments, the first and second temperature sensors are respectfully used to obtain the first temperature and the second temperature at 102 and are arranged as a first sensing module 12 which is secured to the skin of the patient with the first temperature sensor proximal to the patient skin. Similarly, the third temperature sensor and the fourth temperature sensor may be arranged in a second sensing module to obtain the third temperature and the fourth temperature at 104 and configured to be secured to the patient skin with the third temperature sensor proximal to the patient's skin. In such embodiments, the additional values of the two temperature measures obtained at the patient's skin (e.g. from the first temperature sensor and the third temperature sensor) are useful in further determining the body temperature. In a still further exemplary embodiment, the body temperature determined at 114 is a core body temperature located at a position within the patient's body and at an anatomically equal distance from both the first sensor and the third sensor.

In still further exemplary embodiments, the body temperature determined at 114 is wirelessly transmitted across a medical body area network to a patient monitoring device. The patient monitoring device may further be communicatively connected to an electronic medical record of the patient and/or may be connected to a graphical display which presents the determined body temperature.

FIG. 5 is a flow chart that depicts a further detailed exemplary embodiment of a method 200 of measuring body temperature. In an exemplary embodiment, the method 200 may be carried out with a sensor system as described above with respect to FIG. 3, although it will be recognized that other sensor systems may be used while remaining within the scope of the present disclosure.

The method 200 begins at 202 when the sensor detects if the sensor is secured to a patient. In an exemplary embodiment, once the sensor is turned on, the processor monitors the temperature at least one of the temperature sensors, for example the temperature sensor proximal to the patient's skin (e.g. a first temperature sensor), and determines that the sensor has been secured to a patient if the measured temperature shows a consistent increase in the measured temperature. Upon detecting that the sensor is secured to the patient, the processor initiates a calibration process at 204. The calibration process uses a heating device as described herein to create a virtual insulator and establishes a reference measurement of temperatures and resulting heat transfer rates for use in later measurement of the patient's body temperature.

At 206, the processor operates the heating device to begin heating up. In an exemplary embodiment, the heating device is a resistive film secured over the sensor distal from the patient's skin (e.g. a second temperature sensor). The heating device may exemplarily draw power from the battery of the sensor and covert the electrical energy to heat energy. The heating device may be in direct contact with the second temperature sensor or otherwise thermally connected to the second temperature sensor, such that the temperature measured by the second temperature sensor is exemplarily reflective of the temperature of the heating device itself.

At 208, the processor monitors the temperature of the second sensor until a predetermined temperature is reached. In exemplary embodiments, this temperature may be 95° F. In an optional embodiment, the method 200 may continue to monitor the temperatures at the first and second temperature sensors until both of the temperatures are stabilized. In an embodiment, this may be performed at 210 by determining if the temperature at the first temperature sensor is stable. In a non-limiting example, a stable temperature may mean that the temperature has stayed within +/−0.1° F. over the last 10 seconds. If the temperature at the first temperature sensor is not stable, then the processor may continue to 212 and check if the temperature at the second temperature sensor is stable. In an exemplary embodiment, the temperature at the second temperature sensor may be considered stable if the temperature is within +/−0.1° F. the predetermined temperature, e.g. 95° F. If the temperature at the second temperature sensor is not 95+/−0.1° F., then at 214, the power to the heating device is adjusted to achieve the predetermined temperature at the second temperature sensor. Once the temperatures at the first temperature sensor and the second temperature sensors are stable, then the system can measure the temperatures at the sensors.

At 216 a first temperature T₁ is measured by the processor from a signal from the first temperature sensor and the second temperature T₂ is measured by the processor from a signal from the second temperature sensor. These temperatures are stored as the reference temperatures as a result from the calibration phase and can later be used in combination with further temperature measurements as described herein to produce measurements of body temperature.

With completion of the calibration process, the processor further stops the heating device at 218 and the sensor system begins to cool with the removal of this heat source. The system returns to an equilibrium temperature which reflects the heat produced by the body of the patient. In an optional embodiment, at 220, the processor monitors the temperature at the first temperature sensor until it reaches a stable temperature, which may exemplarily be no temperature change within one second or no more than 0.1° F. of temperature change within the last ten seconds, or another definition of stable as may be recognized by a person of ordinary skill in the art.

Next at 222, the processor measures a third temperature T₃ from a signal from the first temperature sensor and a fourth temperature T₄ is measured by the processor from a signal from the second temperature sensor. These temperatures are stored as the measurement temperatures and are used by the processor at 224 to calculate the body temperature. In an exemplary embodiment, the processor calculates the body temperature (T_{C}) from the first, second, third, and fourth temperatures in the manners as described above.

The processor may continue to monitor the temperatures at the first and second temperature sensors and produce a new measurement of body temperature at 226 whenever requested or upon a predetermined temperature measurement interval. To produce the new measurement, the method returns to 222 to acquire a new third temperature and new fourth temperature which may be used in combination with the previously obtained reference first temperature and second temperature to produce the new body temperature measurement.

At 228, a determination may be made to recalibrate the sensor. In an exemplary embodiment this may be made if it is determined that the sensor has been repositioned on the patient, or had been removed and replaced. These determinations may be made based upon a trend in the monitored temperatures at the temperature sensors. In a still further exemplary embodiment, if the body temperature has significantly changed from an initial determination, then it may be determined that recalibration is required to check the validity of the body temperature determination. If a recalibration is required, then the method returns to 204 to repeat the calibration process.

As recognized above, exemplary embodiments of the disclosed systems and methods of measuring body temperature provide a non-invasive solution to monitoring a core body temperature which in some instances may be more representative as an indicator of patient health than other external measurements of body temperature which can vary in their relation to core body temperature based upon size and body composition of the patient. Embodiments of the system and method as described herein can present these advantages in non-invasive measurement of body temperature in a wireless sensor that is suitable for monitoring the condition of the patient in a wireless monitoring system, for example a medical body area network.

In the above description, certain terms have been used for brevity, clarity, and understanding. No unnecessary limitations are to be inferred therefrom beyond the requirement of the prior art because such terms are used for descriptive purposes and are intended to be broadly construed. The different systems and method steps described herein may be used alone or in combination with other systems and methods. It is to be expected that various equivalents, alternatives and modifications are possible within the scope of the appended claims.

The functional block diagrams, operational sequences, and flow diagrams provided in the Figures are representative of exemplary architectures, environments, and methodologies for performing novel aspects of the disclosure. While, for purposes of simplicity of explanation, the methodologies included herein may be in the form of a functional diagram, operational sequence, or flow diagram, and may be described as a series of acts, it is to be understood and appreciated that the methodologies are not limited by the order of acts, as some acts may, in accordance therewith, occur in a different order and/or concurrently with other acts from that shown and described herein. For example, those skilled in the art will understand and appreciate that a methodology can alternatively be represented as a series of interrelated states or events, such as in a state diagram. Moreover, not all acts illustrated in a methodology may be required for a novel implementation.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A method of measuring body temperature, the method comprising:
obtaining a first temperature from a first temperature sensor;
obtaining a second temperature from a second temperature sensor, the first and second temperature sensors separated by a first insulator having a first conductivity value;
obtaining a third temperature;
obtaining a fourth temperature;
calculating a first heat transfer rate from the first temperature and the second temperature;
calculating a second heat transfer rate from the third temperature and the fourth temperature; and
determining a body temperature from the first heat transfer rate and the second heat transfer rate.

2. The method of claim 1, further comprising:
calculating a first differential temperature between the first temperature and the second temperature;
calculating a second differential temperature between the third temperature and the fourth temperature;
wherein the body temperature is determined further based upon the first differential temperature and the second differential temperature.

3. The method of claim 1, wherein the third temperature is obtained from a third temperature sensor and the fourth temperature is obtained from a fourth temperature sensor and the third and fourth temperature sensors are separated by a second insulator having a second conductivity value and the first heat transfer rate is a heat transfer rate between the first sensor and the second sensor across the first insulator and the second heat transfer rate is a heat transfer rate between the third sensor and the fourth sensor across the second insulator.

4. The method of claim 3, wherein the first insulator has a predetermined first thickness and a predetermined first thermal conductivity and the first heat transfer rate is calculated based upon the first thickness and the first thermal conductivity and wherein the second insulator has a predetermined second thickness and a predetermined second thermal conductivity and the second heat transfer rate is calculated based upon the second thickness and the second thermal conductivity.

5. The method of claim 3, wherein the body temperature is a core body temperature common to both the first sensor and the third sensor.

6. The method of claim 5, further comprising positioning the first sensor and the third sensor at equivalent anatomical locations on a patient.

7. The method of claim 3, wherein an adhesive layer is secured across the first and third temperature sensors and a protective layer is secured over the second and fourth temperature sensors, the adhesive layer configured to secure to the skin of the patient, and the protective layer configured reduce exposure of the second insulator and the fourth insulator to wind.

8. The method of claim 1, wherein determining the body temperature further comprises calculating the body temperature based upon the first heat transfer rate, the second heat transfer rate, and the measured first and third temperatures.

9. The method of claim 1, wherein the third temperature is obtained from the first temperature sensor and the fourth temperature is obtained from the second temperature sensor; and
wherein the first and second heat transfer rates are heat transfer rates between the first sensor and the second sensor across the first insulator.

10. The method of claim 9, further comprising:
operating a heating device to increase the first temperature obtained from the first temperature sensor and the second temperature obtained from the second temperature sensor;
after turning off the heating device, obtaining the third temperature from the first temperature sensor and obtaining the fourth temperature from the second temperature sensor.

11. The method of claim 9, wherein the heating first sensor is configured to be proximal the patient's skin from the second sensor and the heating device operates to heat the second sensor to a predetermined target temperature.

12. The method of claim 9, further comprising:
monitoring the first temperature sensor and the second temperature sensor until the temperatures sensed by the first and second temperature sensors stabilize, then obtaining the third temperature measurement and the fourth temperature measurement.

13. The method of claim 9, wherein the body temperature is a first body temperature and further comprising:
obtaining a fifth temperature from the first temperature sensor;
obtaining a sixth temperature from the second temperature sensor;
calculating a third heat transfer rate between the first temperature sensor and the second temperature sensor; and
determining a second body temperature from the first heat transfer rate and the third heat transfer rate.

14. The method of claim 9, wherein an adhesive layer is secured across the first temperature sensor and a protective layer is secured over the second temperature sensor, the adhesive layer configured to secure to the skin of the patient, and the protective layer configured reduce exposure of the first insulator and the second insulator to wind.

15. The method of claim 1, further comprising:
wirelessly broadcasting the determined body temperature with an antenna.

16. A sensing system for measuring body temperature, the sensing system comprising:
a first sensing module comprising:
a first temperature sensor;
a second temperature sensor; and
a first insulator disposed between the first temperature sensor and the second temperature sensor the first insulator having a first conductivity value.

17. The sensing system of claim 16, further comprising:
a second sensing module comprising:
a third temperature sensor;
a fourth temperature sensor; and
a second insulator disposed between the third temperature sensor and the fourth temperature sensor, the second insulator having a second conductivity value wherein the first conductivity value is greater than the second conductivity value.

18. The sensing system of claim 17 further comprising:
a processor communicatively connected to the first, second, third, and fourth temperature sensors, the processor receives measurements of a first temperature from the first temperature sensor, a second temperature from the second temperature sensor, a third temperature from the third temperature sensor, and a fourth temperature from the fourth temperature sensor;
wherein the processor determines a body temperature from a first heat transfer rate between the first temperature sensor and the second temperature sensor across the first insulator calculated from the first temperature and the second temperature and a second heat transfer rate between the third temperature sensor and the fourth temperature sensor across the second insulator calculated from the third temperature and the fourth temperature.

19. The system of claim 17, further comprising
a processor communicatively connected to the first and second temperature sensors, the processor receives measurements of a first temperature and a third temperature from the first temperature sensor, measurements of a second temperature and a fourth temperature from the second temperature sensor;
wherein the processor determines a body temperature from a first heat transfer rate between the first temperature sensor and the second temperature sensor across the first insulator calculated from the first temperature and the second temperature and a second heat transfer rate between the first temperature sensor and the third temperature sensor across the first insulator calculated from the third temperature and the fourth temperature.

20. The system of claim 19, further comprising a heating device disposed distal the second temperature sensor from the first temperature sensor the heating device selectively operable by the processor to heat the first temperature sensor and the second temperature sensor.
